(19) United States
(12) Patent Application Publication
(10) Pub. No.: US 2012/0041022 A1
(43) Pub. Date: Feb. 16, 2012

(54) PROCESS FOR PREPARING ENANTIOMERICALLY ENRICHED ALKALOIDS

(75) Inventors: Lars O. Hansson, Karlskoga (SE); Anders Bergh, Karlskoga (SE)

(73) Assignee: CAMBREX KARLSKOGA, Karlskoga (SE)

(21) Appl. No.: 13/146,547

(22) PCT Filed: Jan. 29, 2010
(86) PCT No.: PCT/GB2010/000143

§ 371 Date: Sep. 30, 2011

Related U.S. Application Data
(60) Provisional application No. 61/202,108, filed on Jan. 29, 2009.

(51) Int. Cl. A61K 31/46; C07D 451/06; C07D 451/02

(52) U.S. Cl. 514/304; 546/132; 546/130; 546/124

(57) ABSTRACT

There is provided a process for the preparation of a single enantiomer of anhydroecgonine (of formula I):

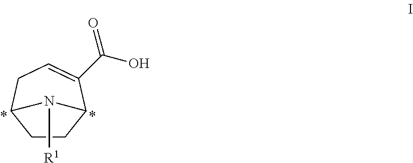

or a salt thereof, in which R¹ is as defined in the description. Such single enantiomers may, for example, be useful intermediates in the synthesis of pharmaceuticals, in which the enantioselectivity is important.

The present invention relates to processes for the preparation of certain alkaloids, for example those based on a tropane bicyclic structure, in an enantioselective manner.

International patent applications WO 96/30371, WO 02/102801 and WO 2004/072071 disclose a method for the preparation of the alkaloid anhydroecgonine (ecgonidine), which process comprises the elimination of the corresponding tropane derivative containing a carboxy and hydroxy moiety (or a derivative thereof). The elimination is effected in the presence of an alkoxide salt (such as sodium ethoxide).

A process for the preparation of a single enantiomer of anhydroecgonine (the R- or L-enantiomer) from cocaine is also know, which process can be effected by the treatment of cocaine in a strong aqueous acid in order to promote an elimination step. Such a process is described in de Jong, Recl. Tray. Chim. Pays-Bas, 56 (1937), pages 187-201, which journal article is referenced in international patent application WO 96/30371.

Journal article by Bell et al, J. Am. Chem. Soc., 82, 4642-4644, 1961 discloses the synthesis of an apparently unisolated single enantiomer of anhydroecgonine from cocaine hydrochloride, which is refluxed in aqueous hydrochloric acid for several hours to yield the 2-carboxytropin-3-ol (L-ecgonine hydrochloride). Thereafter an elimination reaction occurs in the presence of phosphorous oxychloride to yield L-anhydroecgonine as an unisolated intermediate in the formation of a corresponding amide.

Journal article by Majewski et al, J. Org. Chem. 1995, 60, 5825-5830 also discloses the synthesis of a single enantiomer of an ester of anhydroecgonine. The synthesis starts from the enantioselective deprotonation of 3-tropinone and methoxycarbonylation to form a single enantiomer of 2-carboxymethyltropinone. Thereafter, catalytic hydrogenation and elimination of 2-carboxymethyltropin-3-ol (in the presence of base and trifluoroacetic anhydride) afforded a single enantiomer of anhydroecgonine methyl ester (an ee of 94% for the methyl ester of the (+)-enantiomer is reported to be obtained). This journal article only discloses a basic elimination to form a single enantiomer of anhydroecgonine.

Journal article by Atkinson et al, J. Org. Chem., Vol. 36, No. 21, 1971 discloses the synthesis of an amide derivative of racemic anhydroecgonine (which presumably proceeds via anhydroecgonine itself, which intermediate is not isolated), from a 2-carboxytropin-3-ol followed by an elimination reaction. The 2-carboxytropin-3-ol is produced via reduction by hydrogenation of corresponding 2-carboxymethyltropinone, which (due to the selective nature of the catalytic hydrogenation, i.e. the delivery of hydrogen onto only one face of the keto group) produces only two possible diastereoisomers around the 2- and 3-positions of the tropinol produced. The two possible diastereoisomers that are produced are the cis ones (i.e. those in which the carboxy and hydroxy groups at the 2- and 3-positions are on the same face), which are eliminated under the conditions described in the above-mentioned journal article by Bell et al, J. Am. Chem. Soc., 82, 4642-4644, 1961.

Journal article by Zirkle et al, J. Org. Chem., 27, 1269-1278, 1962 also discloses the synthesis of anhydroecgonine methyl ester via an elimination of 3-carboxy-tropin-3-ol. This journal article also mentions the synthesis of anhydroecgonine from the elimination of ecgonine in the presence of phosphorous oxychloride or hydrochloric acid, as described in the de Jong journal article mentioned above.

Furthermore, none of the above-mentioned documents disclose the preparation of a solid crystalline form of a salt of a single enantiomer of anhydroecgonine. The only disclosure in the literature of a crystalline form of the hydrogen chloride salt of anhydroecgonine is one in which the hydrogen chloride salt is further converted into a gold salt (e.g. AuCl₃) (see for example, A. Einhorn, Ber., 20, 1221 (1887) and Merck et al, Diese Berichte XIX, 3002 (1886)).

The formation of a particular crystalline salt of a compound may be advantageous (as compared to, for example, an amorphous form), as crystalline forms may be easier to purify and/or handle. Further, when the desired crystalline product is a single enantiomer, then the enantiomeric excess may be improved by recrystallisation (the minor enantiomer remaining in the mother liquor). Crystalline forms may also have a better solid state stability and shelf-life (e.g. be stored for longer periods of time without substantial change to the physico-chemical characteristics, e.g. chemical composition, density and solubility).

The skilled person will appreciate that, if a compound can be obtained in stable crystalline form, then several of the above-mentioned disadvantages/problems with amorphous forms may be overcome. Furthermore, in the case of anhydroecgonine hydrochloride, the conversion to a crystalline form as described in journal articles A. Einhorn, Ber., 20, 1221 (1887) and Merck et al, Diese Berichte XIX, 3002 (1886) is achieved by conversion to the AuCl₃ salt, which is an expensive and impractical method. There is therefore a real practical need for a more reliable process.

It should be noted that obtaining crystalline forms is not always achievable, or not easily achievable. Indeed, it is typically not possible to predict (e.g. from the molecular structure of a compound), what the crystallisation behaviour of a certain compound, or a salt of it, may be. This is typically only determined empirically.

There is a need for alternative processes for the preparation of either enantiomer of anhydroecgonine, especially in high enantiomeric excess and/or in a better level of purity.

The listing or discussion of an apparently prior-published document in this specification should not necessarily be taken as an acknowledgement that the document is part of the state of the art or common general knowledge.

There is now provided a process for the preparation of a single enantiomer of anhydroecgonine (of formula I):

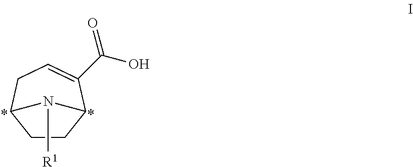

or a salt thereof, wherein:

R¹ represents hydrogen or optionally substituted C₁₋₁₂ alkyl;

the asterisks each denote a chiral centre that has a certain configuration, by elimination (and, if required, hydrolysis) of a compound of formula II,

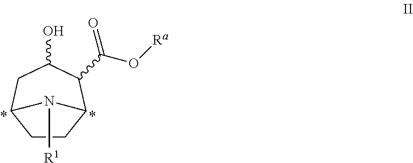

wherein:

the asterisks each denote a chiral centre that has a certain configuration;

the squiggly lines (attached to the OH group and to the C(O)OR^{a} group) each denote a bond that is attached to a chiral centre that can be of R or S-configuration;

R^{a} represents: hydrogen; optionally substituted aryl or heteroaryl; or, preferably, optionally substituted C₁₋₁₂ alkyl; and

R¹ is as defined above,

in the presence of a strong acid and a carboxylic acid,

hereinafter referred to as “the process of the invention”.

The process of the invention may be performed employing salts or solvates (or protected derivatives) of the compound of formula II. Compounds of formula I that may thereby be produced may or may not be produced in the form of a (e.g. corresponding) salt or solvate (or protected derivative). Salts of compounds of formula I may also be produced by virtue of association with the reactants (e.g. the strong acid to form an acid salt). In any event, if a salt of the compound of formula I is desired from the process of the invention (or from a separate step after the process of the invention), the skilled person will appreciate what conditions are required. Further if the free base of a compound of formula I is required, and the process of the invention results in the formation of a salt (e.g. a salt that is an association of the compound of formula I and an acid, such as the strong acid employed in the process) then the skilled person will appreciate that neutralisation under standard condition (e.g. those described herein) may need to be effected. Where a “compound of formula I” is referred to herein, the reference may be to the free base of a compound of formula I or to a salt thereof.

It is stated herein that a single enantiomer of the compound of formula I is formed from the process of the invention. The process of the invention may be employed to form either the R- or the S-enantiomer, dependent upon the configuration of the starting material (the compound of formula II) that is employed (which in turn depends upon the configuration of the precursor compound of formula III, as described hereinafter, to be formed). By single enantiomer, we mean that the enantiomeric excess is greater than 50%, i.e. there is more of one enantiomer than the other. Preferably, we mean that the enantiomeric excess is greater than 60%, more preferably greater than 70%. Particularly preferred are enantiomeric excesses greater than 80%, especially greater than 90%. Most preferably, the enantiomeric excess is close to 100% (i.e. greater than 95%, for example greater than 99%), with a negligible amount of the minor enantiomer.

It is stated herein that asterisks denote a chiral centre that has a certain configuration. That is, the relevant chiral centres may be either in the R- or S-configuration. In line with the definition of ‘single enantiomer’ above, the asterisk denotes a chiral centre that is either predominantly in the R-configuration (i.e. the R-enantiomer) or the S-configuration (i.e. the S-enantiomer). Preferably, either the R- or the S-configuration is present in a ratio (of one to the other) of greater than 60:40, preferably greater than 70:30 (e.g. greater than 80:20, especially greater than 90:10). Most preferably, either the R- or the S-configuration is present in a ratio close to 100:0 (i.e. greater than 95:5, for example greater than 99:1), with a negligible amount of the minor R- or S-configuration.

It is stated herein that the squiggly line (attached to the OH group or the C(O)OR^{a} group) denotes a bond which is attached to a chiral centre that can be of R- or S-configuration. Therefore, depending on the nature of the compound of formula II, i.e. if both the OH group and the C(O)OR^{a} group have a certain configuration, then the compound of formula II employed in the process of the invention may be a single enantiomer and single diastereoisomer. Alternatively, depending upon the configuration of the OH group and the C(O)OR^{a} group, the compound of formula II employed in the process of the invention may be another relevant enantiomer and/or diastereoisomer. The compound of formula II may exist as a mixture of four diastereoisomers, as explained hereinafter.

It is stated above that the relevant enantiomers and/or diastereoisomers of the compound of formula II may be employed in the process of the invention. In this regard, the stereochemistry of the compound of formula II may, for example, be controlled in the synthesis to obtain the compound of formula II (for example, the precursor such as the compound of formula III may possess a certain stereochemistry, e.g. be a certain enantiomer and/or diastereoisomer, or, the stereochemistry of the compound of formula II may be controlled by the process step to obtain it, e.g. by the use of reagents that provide selectivity).

Although the process of the invention may be employed using any of the relevant enantiomers/diastereoisomers of the compound of formula II, it is preferred that, in the compound of formula II, each chiral centre attached to a squiggly line is a mixture of R- and S-configurations. The mixture of configurations may emanate from the mixture of configurations at the chiral centre to which the —C(O)OR^{a} group is attached in any precursor to the compound of formula II (e.g. in the compound of formula III) and/or from the non-selective reaction of any precursor to the compound of formula II (e.g. from the non-selective reduction of the keto group of a compound of formula III). By “non-selective”, we mean that there is not complete stereoselectivity (or stereospecificity), for example a non-selective reaction may not substantially bias the stereochemistry of the product to be formed (e.g. it may produce a ratio of less than 90:10, for instance less than 70:30, and may be near to a ratio of 50:50 of possible stereoisomers). Hence, each chiral centre to which the OH and C(O)OR^{a} group is attached may each bear those respective groups in a mixture of the two R- and S-configurations, for instance, a mixture of less than 90:10, for example less than 70:30 and may be an approximately equal mixture; i.e. a ratio of about 50:50 of one configuration to the other). Hence, in the compound of formula II, a mixture of the two R- and S-configurations at each of the chiral centres to which the —OH group and —C(O)OR^{a} group are respectively attached, would give rise to four possible diastereoisomers (i.e. the (R,R), (R,S), (S,R) and (S,S) diastereoisomers). However, the compound of formula II, although it may exist as a mixture of four diastereoisomers, this is not an essential feature. Only certain diastereoisomers (e.g. any three or less, e.g. two or even any one) of the compound of formula II may be present for the process of the invention to be performed.

Compounds employed in or produced by the processes of the invention described herein may exhibit tautomerism. The process of the invention therefore encompasses the use or production of such compounds in any of their tautomeric forms, or in mixtures of any such forms.

Unless otherwise specified by an asterisk or squiggly line, the compounds employed in or produced by the processes of the invention described herein may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Further, unless already specified (for example if the double bond is contained in a ring), the compounds employed in or produced by the processes of the invention described herein may contain double bonds and may thus exist as E (entgegen) and Z (zusammen) geometric isomers about each individual double bond. All such isomers and mixtures thereof are included within the scope of the invention.

Unless otherwise specified, alkyl groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl groups may also be part cyclic/acyclic. Such alkyl groups may also be saturated or, when there is a sufficient number (i.e. a minimum of two) of carbon atoms, be unsaturated.

The term “aryl”, when used herein, includes C₆₋₁₄ (e.g. C₆₋₁₀) groups. Such groups may be monocyclic, bicyclic or tricyclic and, when polycyclic, be either wholly or partly aromatic. C₆₋₁₄ (e.g. C₆₋₁₀) aryl groups that may be mentioned include phenyl, naphthyl, and the like. For the avoidance of doubt, the point of attachment of substituents on aryl groups may be via any carbon atom of the ring system.

The term “heteroaryl”, when used herein, includes 5- to 14-membered heteroaryl groups containing one or more heteroatoms selected from oxygen, nitrogen and/or sulfur. Such heteroaryl group may comprise one, two or three rings, of which at least one is aromatic. Substituents on heteroaryl groups may, where appropriate, be located on any atom in the ring system including a heteroatom. The point of attachment of heteroaryl groups may be via any atom in the ring system including (where appropriate) a heteroatom. Examples of heteroaryl groups that may be mentioned include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, quinolinyl, benzoimidazolyl and benzothiazolyl.

The term “halo”, when used herein, includes fluoro, chloro, bromo and iodo. Isotopes of halo atoms may be included.

Advantageously, the elimination reaction of the process of the invention may produce a compound of formula I, starting from any of the four possible diastereoisomers of the compound of formula II (or mixtures thereof). Hence, if mixtures of diastereoisomers are produced in a process to obtain the compound of formula II, then those diastereoisomers need not be separated before the reaction to obtain the compound of formula I and/or certain diastereoisomers may not be left unreacted by the process of the reaction or be converted into undesired side-products. Rather, the process of the invention does not require certain diastereoisomers of the compound of formula II to be produced, for conversion to the compound of formula I to take place, for example as may be the case for reaction conditions and processes described in the prior art.

For example, in the instance where base-induced eliminations have been performed in the prior art, such elimination reactions may require precursors in which the 2-carboxymethyl moiety and 3-hydroxy moiety are cis (i.e. on the same face) with respect to each other. This is because the hydrogen atom attached to the carbon atom bearing the carboxymethyl group may have to be substantially anti-periplanor to the leaving group (i.e. the hydroxy group, which is appropriately converted into a better leaving group) in order for the E2 type elimination to be effected. In such methods involving base-eliminations, only single diastereoisomers of the 2-carboxymethyltropin-3-ol may be produced (in which the 2-carboxymethyl moiety and 3-hydroxy moiety are cis with respect to each other). In any event, only a maximum of two of the four possible diastereoisomers (i.e. the two possible cis diastereoisomers) that may be present due to the chiral centres at the 2- and 3-positions of the tropinol (the skilled person will appreciate that additional diastereoisomers may be possible due to the additional chiral centres in the tropinol, e.g. at the 1-position) may be eliminated via this method to produce a single enantiomer of anhydroecgonine.

Furthermore, in the instance where acid-based eliminations have been effected in the prior art in the presence of HCl (in the absence of any other acid), then these conditions may not be suitable to eliminate all four diastereoisomers.

As stated above, the elimination step of the process of the invention occurs in the presence of a strong acid and carboxylic acid, which may help to promote the elimination step of the process of the invention. For instance, the carboxylic acid may react with the compound of formula II to form an acylated intermediate of formula IIA,

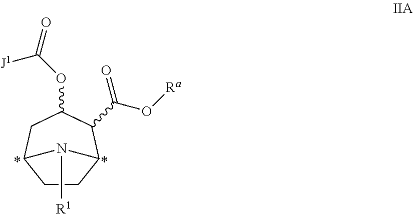

in which J¹ represents the counterpart to the carboxylic acid functional group, and R^{a}, R¹, the squiggly lines and the asterisks are as hereinbefore defined (but R^{a} preferably represents hydrogen; which may be formed in situ by hydrolysis of the corresponding ester, i.e. of a corresponding compound in which R^{a} does not represent hydrogen). Such intermediates that may be formed may advantageously increase the rate of reaction, for example by providing a better leaving group for the elimination reaction to take place.

In the process of the invention, it is stated that a carboxylic acid is employed, i.e. an organic acid characterised by the presence of a —COOH group, e.g. a compound of formula IIB,

J¹-COOH IIB

in which J¹ is the counterpart to the carboxylic acid functional group. For example, J¹ may represent hydrogen or C₁₋₁₂ (e.g. C₁₋₆) alkyl, which is optionally substituted by one or more substituents selected from carboxy (e.g. —COOH), halo (e.g. fluoro) and aryl (e.g. phenyl; optionally substituted by one or more halo (e.g. fluoro, chloro or bromo) atoms). Preferably, J¹ represents unsubstituted C₁₋₃ (e.g. C₁₋₂) alkyl (e.g. methyl or ethyl), so forming, for example, acetic acid or, preferably, propionic acid.

In the process of the invention, a strong acid is employed. By strong acid, we refer to an acid that ionises in water to a substantial degree (for example an acid that ionises to near 100%). Preferably, we refer to a strong inorganic acid, such as any suitable mineral acid, or suitable salts thereof (for example, phosphoric acid, nitric acid, sulfuric acid, or salts thereof, such as sodium hydrogen sulphate, or, more preferably, a hydrogen halide acid, e.g. HCl, HI, or, most preferably, HBr). Preferably, the strong acid is a hydrogen halide, especially, HBr.

In the process of the invention, the strong acid may be added to the compound of formula II. Preferably, at least one molar equivalent of strong acid (e.g. hydrogen halide, such as HBr) is added, for example, about 1.5 equivalents. The mixture may be reacted at elevated temperature, for example at reflux.

The strong acid may be employed at any suitable concentration by weight (provided that a sufficient molar quantity is employed). However, preferably, it is employed as a solution containing at least 10% (e.g. at least 20%, e.g. at least 30%, up to e.g. about 60%) of strong acid (e.g. hydrogen halide, such as HBr) by weight. More preferably, the strong acid is employed as a solution containing between 40 and 60% (e.g. between 45 and 55%, e.g. about 48%) by weight of the strong acid.

In the process of the invention, the carboxylic acid may also be added to the compound of formula II (or mixture of compound of formula II and strong acid). Preferably, at least one equivalent of carboxylic acid (e.g. proprionic acid) is added, for example, at least 1.5 equiv. Preferably, at least two or at least three molar equivalents are added (e.g. about five molar equivalents). The carboxylic acid component of the process of the invention may serve as a solvent. Optionally, the process of the invention may be performed in the presence of a further solvent (or mixture of solvents).

The mixture of the carboxylic acid and strong acid may form an azeotropic mixture, which is easy to handle in the process of the invention. However, any azeotropic mixture may provide a medium that is difficult to separate any product formed by the process of the invention (e.g. by distillation), unless the medium is suitable for precipitation of the product and/or the nature of the product itself (e.g. if it is a crystalline form) results in its precipitation from the reaction mixture, which is clearly advantageous in terms of separation and/or purification of any product formed.

In the process of the invention, the mixture of the compound of formula II, strong acid and carboxylic acid may be reacted (together with any other components that may be present, e.g. any optional further solvents), preferably at elevated temperature, for example at above 100° C. (e.g. at above 150° C., e.g. at about 165° C.±5° C.). However, the temperature of the reaction mixture may be dependent upon the boiling point of the carboxylic acid and/or other solvent that may be present in the mixture. The reaction may be monitored for the presence of starting material, and the reaction time may consequently be adjusted (e.g. lengthened, if there the presence of starting material is indicated). The temperature of the reaction may also be adjusted, depending on whether it is desired to adjust the rate of reaction.

In a further aspect of the invention, there is provided either:

(A) a crystalline compound formed by an association between:
(i) a compound of formula I (as hereinbefore defined); and
(ii) an acid; and/or

(B) a compound formed by an association between:
(i) a compound of formula I (as hereinbefore defined); and
(ii) HI or, preferably, HBr,
   which compounds are referred to herein as “the compounds of the invention”.

Compounds of the invention are formed by an association between moieties (i) and (ii). For the avoidance of doubt therefore, compounds of the invention comprise an association between moieties defined by (i) and (ii) above.

The aforementioned association between moieties (i) and (ii) may be any kind of physico-chemical association (i.e. interaction or bonding) between the respective moieties, for example an ionic association (wholly or in part), so forming a salt, or one or more other kinds of association (wholly or in part), such as a covalent (including polar covalent and coordinate covalent) association, a metallic association, or another, electrostatic association, such as a permanent dipole to permanent dipole interaction, hydrogen bonding, van der Waals forces and/or a cation-pi interaction. It is however preferred that the association is at least partly ionic, so forming a salt.

In the case of (A) above (and preferably, for the case of (B) above), the association (e.g. salt) so formed is one in which there is a 1:1 ratio (or thereabouts, e.g. between 1.5:1 and 1:1.5 such as between 1.2:1 and 1:1.2) of the moiety (i) (i.e. the compound of formula I) and moiety (ii) (i.e. the acid). That is, in the case of the formation of a salt, (A) is essentially a crystalline compound (or (B) is essentially a compound) of the following formula:

(Compound of formula I)ₘ×(acid)ₙ

wherein m and n represent numerical values in which m/n is between 3/2 and 2/3 (for example, between 6/5 and 5/6, and most preferably, about 1). In the case of (B), in the above formula, the acid is either HI or HBr. Factors such as the degree of ionisation may affect the actual values.

Compounds of the invention may be obtained in forms which are greater than 80% ionic (i.e. in salt form). However, by “at least partly ionic” we include greater than 20%, preferably greater than 30%, and more preferably greater than 40% ionic. The degree (%) of ionisation may be determined by the skilled person using standard techniques, such as solid state NMR, FT-IR, Raman spectroscopy, X-ray diffraction, differential scanning calorimetry (DSC) and microcalorimetry.

Hence in further aspects of the invention, there is provided:

(A) a crystalline acid addition salt of a compound of formula I, characterised in that it consists essentially of the compound of formula I and the salt in a ratio of 1:1 (i.e. a crystalline mono-acid salt of a compound of formula I); and/or

(B) a HI or, preferably a HBr salt of a compound of formula I.

Preferably, the acid component (ii) in the (A) embodiments described above (e.g. the acid addition salt of the crystalline acid addition salt of the compound of formula I) is a hydrogen halide, e.g. HCl, HI or most preferably, HBr.

Preferably, in the (B) embodiments described above, the compound formed (i.e. the association of a compound of formula I and HI or HBr, e.g. a HI or HBr salt salt) is preferably in solid form. More preferably, it is in solid crystalline form. Preferably, it is in a form in which the compound of formula I and the HI or HBr are in a ratio of about 1:1 (e.g. a mono-HI or mono-HBr salt of a compound of formula I may be formed. In such instances, some of the preferred embodiments described here (in respect of (B) embodiments) may also be (an) embodiment(s) described by (A) above.

By “crystalline”, we mean in substantially crystalline form, by which we include forms that are greater than 10%, e.g. greater that 20%, preferably greater then 30%, and more preferably greater than 40% crystalline. Most preferably, the forms are greater than 50%, e.g. greater than 80% or preferably greater than 90% crystalline. The degree (%) of crystallinity may be determined by the skilled person using X-ray powder diffraction (XRPD). Other techniques, such as solid state NMR, FT-IR, Raman spectroscopy, differential scanning calorimetry (DSC) and microcalorimetry, may also be used.

It is stated herein that crystalline forms may have a better properties relating to stability, e.g. when stored under normal storage conditions. The term “stability” as defined herein includes chemical stability and solid state stability.

By “chemical stability”, we include that the compound, or salt, can be stored in an isolated form, under normal storage conditions, with an insignificant degree of chemical degradation or decomposition.

By “solid state stability”, we include that the compound, or salt, can be stored in an isolated solid form, under normal storage conditions, with an insignificant degree of solid state transformation (e.g. crystallisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, such as 15 to 30° C.), pressures of between 0.1 and 2 bars (preferably at atmospheric pressure), relative humidities of between 5 and 95% (preferably 10 to 75%), and/or exposure to 460 lux of UV/visible light, for prolonged periods (i.e. greater than or equal to six months). Under such conditions, compounds of the invention may be found to be less than 15%, more preferably less than 10%, and especially less than 5%, chemically degraded/decomposed, or solid state transformed, as appropriate. The skilled person will appreciate that the above-mentioned upper and lower limits for temperature, pressure and relative humidity represent extremes of normal storage conditions, and that certain combinations of these extremes will not be experienced during normal storage (e.g. a temperature of 50° C. and a pressure of 0.1 bar).

There are further provided processes for the preparation of compounds of the invention (e.g. described by embodiments (A) and (B) above).

For example, there is further provided a process for the preparation of compounds of the invention (i.e. a crystalline compound formed by the association of a compound of formula I with an acid or a solid compound formed by the association of a compound of formula I with HI or, preferably, HBr; wherein in both cases the association preferably results in a salt), which comprises:

(i) bringing into association (or “contacting”) a compound of formula I and the acid (e.g. HI or, preferably HBr) in a solvent;

(ii) crystallisation or precipitation of the compound (e.g. the crystalline acid addition salt, or, solid HI or HBr salt) so formed, in the solvent.

The process for the preparation of compounds of the invention described above may have the advantage that the crystallisation or precipitation step results in the production of the compound that is in a form that is more pure than corresponding or similar compounds prepared in the prior art (for example, as compared to solid forms obtained by evaporation, which method may leave residual impurities). Hence, in a further aspect of the invention, there is provided compounds (e.g. compounds of the invention or compounds formed by the process of the invention) obtainable by the processes described herein.

More specifically, there is further provided a process for the preparation of the crystalline acid addition salt of the compound of formula I (see embodiments (A) above), which comprises:

(i) preparation of the acid addition salt of a compound of formula I (for example, as defined herein) in a solvent;

(ii) crystallisation of the acid addition salt so formed, in the solvent.

Crystallisation (or precipitation) of the compounds of the invention (e.g. the acid addition salt of the compound of formula I) may be performed in any suitable solvent (or mixtures of solvents). Solvents that may be employed in the reaction mixture may be, for example, an aqueous solvent (although this is preferably removed or reduced in volume prior to crystallisation/precipitation), a C₁₋₆ alcohol (e.g. isopropanol or ethanol) or, preferably, is weak organic acid (such as a carboxylic acid as defined herein, e.g. formic, acetic or propionic acid). Preferably, the crystallisation solvent is homogenous, for example the solvents may forms an azeotropic mixture. However, a suitable solvent may also be employed as an “anti-solvent” (i.e. a solvent in which salts of compounds of formula I are poorly soluble) in order to aid the crystallisation process.

Crystallisation temperatures and crystallisation times depend upon the concentration of the compound in solution, and upon the solvent system which is used.

Crystallisation may also be initiated and/or effected with or without seeding with crystals of the appropriate crystalline compound of the invention, and/or by adjustment of pH.

Advantageously, when a hydrogen halide (e.g. HCl, preferably, HI or, especially HBr) is employed as the strong acid in the process of the invention to obtain compounds of formula I, then during the process, there may be the necessary association between the compound of formula I and the acid (e.g. the HBr salt) to form the compounds of the invention (i.e. those outlined by (A) and (B) above, e.g. the salts). That is, compounds of the invention (e.g. those described by embodiments (A) and (B) above) may be prepared directly from the process of the invention (i.e. the process which comprises elimination of the compound of formula II to form a compound of formula I, or a salt thereof).

More advantageously still, the reaction medium (e.g. the mixture of aqueous solution of strong hydrogen halide, e.g. HBr, acid and carboxylic acid) in the process of the invention may promote crystallisation (or precipitation) of the compounds of the invention (e.g. the hydrogen halide (e.g. HBr) salt of the compound of formula I). This may aid the purification of the compounds of the invention or the product of the process of the invention as it may crystallise/precipitate out of solution. Hence, the product obtained need not be purified and/or separated by other means (such as by distillation).

Further still, the formation of a crystalline salt of a compound of formula I may also allow the improvement of enantiomeric excess in the product by means of standard recrystallisation techniques, which may not be possible when the product is not in crystalline form. The possibility of increasing the enantiomeric excess in this manner may also mean that in the process of the invention, the enantiopurity (or ee) of the starting material (i.e. compound of formula II or any precursor thereof, e.g. compound of formula III) may be compromised, i.e. any starting materials need not have a high ee, in order to obtain high ees in any product formed. For example, the compound of formula II or III may have an ee of down to 90%, and even an ee of down to 80% (or less).

Preferably, the strong acid employed in the process of the invention may serve to form (i.e. may be moiety (ii)) compounds of the invention (as defined by (A) and (B) above), for example, a salt of the compound of formula I (e.g. a hydrogen halide salt, e.g. HBr salt), which is preferably in solid crystalline form.

In yet a further aspect of the invention, there is provided a product obtainable by the processes described herein. For example, there is provided a single enantiomer of any of the relevant compounds described herein (e.g. compound of formula I, IA, IB, IC or ID), e.g. anhydroecgonine (for example the (−)-enantiomer or, preferably, the (+)-enantiomer) characterised in that:

(i) the ee is greater than 95% (e.g. greater than 98%, e.g. greater than 99%, such as near to 100% ee); and/or

(ii) the product is in a form that is more pure (e.g. contains less impurities, including the undesired enantiomer), for example a HPLC (chiral or achiral) purity of greater than 95%, e.g. 98% (preferably greater than 99%).

The compounds of formula I, or salts thereof (as well as any downstream products), obtained by the process of the invention may be separated and/or isolated by standard techniques, for instance by chromatography, crystallisation, distillation, evaporation of solvents and/or by filtration.

In the process of the invention, it is preferred that:

when R^{a} represents optionally substituted aryl or heteroaryl, then the optional substituents are preferably selected from halo and C₁₋₃ alkyl (however, such aryl or heteroaryl groups are preferably unsubstituted);

when R^{a} represents optionally substituted C₁₋₁₂ alkyl, then the optional substituents are preferably selected from fluoro (however, such alkyl groups are preferably unsubstituted);

particularly preferred R^{a} groups include C₁₋₃ (e.g. C₁₋₂) alkyl groups such as methyl or, preferably, ethyl.

In the process of the invention, it is further preferred that:

when R¹ represents optionally substituted C₁₋₁₂ alkyl, then that group may be optionally substituted by one or more substituents selected from halo (e.g. iodo or, preferably, fluoro) and ═O;

R¹ preferably represents hydrogen or C₁₋₆ alkyl (e.g. C₁₋₃ alkyl) optionally substituted by one or more substituents selected from ═O, iodo, and, preferably, fluoro;

when R¹ represents a C₁₋₁₂ alkyl group substituted by ═O at the position adjacent the requisite nitrogen atom (in the compound of formula I, II, etc) then the skilled person will appreciate that an amide moiety may be formed (i.e. R¹ may represent —C(O)—C₁₋₁₁ alkyl, in which the C₁₋₁₁ alkyl moiety is optionally substituted with the substituents defined herein, e.g. fluoro, but the C₁₋₁₁ alkyl moiety is preferably unsubstituted;

R¹ represents hydrogen or C₁₋₆ (e.g. C₁₋₃) alkyl (optionally substituted by one or more substituents selected from ═O, preferably, iodo and, more preferably fluoro (so forming for example a 3-iodo-3-propenyl (i.e. —CH₂—CH═CH—CH₂l ) or, preferably a 3-fluoro-propyl (i.e. —CH₂)₃—CH₂F) group);

R¹ most preferably represents methyl.

In a further aspect of the invention, the compound of formula I, or salts thereof, may be further modified by derivatisation (e.g. by forming an ester, amide or ether). If necessary, if a salt of the compound of formula I (or other compounds of the invention, e.g. those described in embodiments (A) and (B) above), are formed by the process of the invention, then the salt may first need to be neutralised (e.g. under conditions described herein) before further modification of the compound of the invention/compounds of formula I.

Hence, in a further aspect of the invention, the compound of formula I, or salt thereof, may further be converted to the following compound of formula IA,

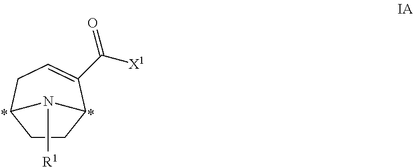

in which X¹ represents:

(i) —OR^{b1};

(ii) —N(R^{b2})R^{b3},

R^{b1} represents optionally substituted aryl or heteroaryl (in which the optional substituents are preferably selected from halo and C₁₋₃ alkyl; but such groups are preferably unsubstituted) or, preferably, optionally substituted C₁₋₁₂ (e.g. C₁₋₆) alkyl (in which the optional substituents are preferably selected from fluoro; but such groups are preferably unsubstituted). Particularly preferred R^{b1} groups include unsubstituted C₁₋₄ (e.g. C₁₋₂) alkyl groups, such as methyl or, preferably, ethyl;

R^{b2} and R^{b3} independently represent optionally substituted aryl or heteroaryl (in which the optional substituents are preferably selected from halo and C₁₋₃ alkyl; but such groups are preferably unsubstituted) or, preferably, hydrogen; optionally substituted C₁₋₁₂ (e.g. C₁₋₆) alkyl (in which the optional substituents are preferably selected from fluoro; but such groups are preferably unsubstituted);

R¹ is as hereinbefore defined.

For the conversion to compounds of formula IA in which X¹ represents —OR^{b1}, standard esterification conditions may be performed on compounds of formula I, in the presence of a compound of formula IAA,

R^{b1}—OH IAA

wherein R^{b1} is as hereinbefore defined, for example in acidic reaction conditions, e.g. in the presence of a strong acid, such as a protic acid, e.g. sulfuric acid. The esterification may be performed in the presence of solvent, in which, preferably, the reactant compound of formula IAA serves as the solvent itself. The esterification may be performed at elevated temperature, for example at the reflux temperature of the compound of formula IAA.

For the conversion to compounds of formula IA in which X¹ represents —N(R^{b2})R^{b3}, standard amide coupling reactions may be performed on compounds of formula I, which may be reacted in the presence of a compound of formula IAB,

HN(R^{b2})R^{b3} IAB

wherein R^{b2} and R^{b3} are as hereinbefore defined, for example in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof) or N,N′-disuccinimidyl carbonate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, the carboxylic acid group of the compound of formula I may first be converted under standard conditions to the corresponding acyl chloride (e.g. in the presence of POCl₃, PCl₅, SOCl₂ or oxalyl chloride), which acyl chloride is then reacted with a compound of formula IAB, for example under similar conditions to those mentioned above.

The compound of formula I, or salt thereof, or, a compound of formula IA in which X¹ represents —OR^{b1}, may also be converted to a compound of formula IB,

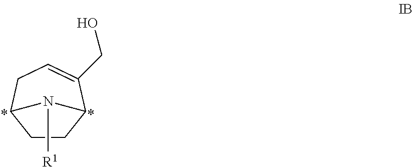

wherein R¹ is as hereinbefore defined, by an appropriate reduction. For example for the reduction of the ester of formula IA (in which X¹ represents —OR^{b1}), preferred reducing agents include lithium aluminium hydride and lithium borohydride. For the reduction of the carboxylic acid of formula I, preferred reducing agents include borane (or complexes) thereof. A similar reaction may be performed on a compound of formula IA in which X¹ represents —OR^{b1} to obtain compounds of formula IB, employing suitable reduction conditions (e.g. reaction in the presence of a reducing agent such as lithium aluminium hydride or the like).

In a further aspect of the invention, compounds of formula IB may be derivatised to form a compound of formula IC,

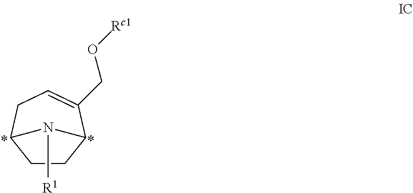

wherein R^{c1} represents optionally substituted C₁₋₁₂ (e.g. C₁₋₆) alkyl (in which the optional substituents are preferably selected from fluoro; but such groups are preferably unsubstituted), and R¹ is as hereinbefore defined. Such a conversion may be performed under standard alkylation reaction conditions in the presence of a compound of formula ICA,

L¹-R^{c1} ICA

wherein L¹ represents a suitable leaving group, such as chloro, bromo, iodo, a sulfonate group (e.g. —OS(O)₂CF₃, —OS(O)₂CH₃, —OS(O)₂PhMe or a nonaflate), and R^{c1} is hereinbefore defined, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine).

In a further aspect, the compounds of formula I (or e.g. compounds of formula IA, IB or IC) may be converted to other compounds of formula I (or e.g. compounds of formula IA, IB or IC). For example, compounds of formula I in which R¹ represents —C(O)C₁₋₁₁ alkyl or, e.g. methyl, may be converted to corresponding compounds of formula I in which R¹ represents hydrogen (for example, under standard conditions known to those skilled in the art, e.g. to effect the cleavage of the relevant R¹ group). Alternatively, compounds of formula I in which R¹ represents hydrogen may be converted to compounds of formula I in which R¹ represents C₁₋₁₂ alkyl, for example under standard conditions, e.g. alkylation or (when R¹ represents —C(O)C₁₋₁₁ alkyl), acylation reaction conditions.

In a further aspect, the compounds of formula I (or compounds of formula IA, or derivatives thereof, e.g. in which the R¹ moiety has been modified under conditions that may have been described herein) may be converted to other compounds of formula ID,

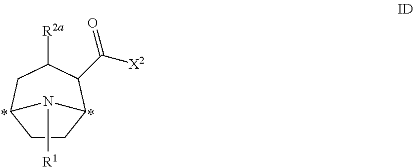

wherein X² represents —OH (in the case of modification of compounds of formula I) or X¹ (in the case of modification of compounds of formula IA; wherein X¹ is as hereinbefore defined), R^{2a} represents heteroaryl or, preferably, aryl (e.g. phenyl) optionally substituted by one or more substituents selected from halo (or an isotope thereof) and C₁₋₃ alkyl (optionally substituted by halo or an isotope thereof), by reaction of a corresponding compound of formula IDA,

R^{2a}—Mg-halo IDA

or the like (i.e. a Grignard reagent formed under standard conditions), wherein R^{2a} is as hereinbefore defined and which Michael addition reaction is performed under standard conditions.

The carboxylic acid group of the compound of formula I (or any relevant product downstream, e.g. compound of formula IB, IC, ID) may be esterified or derivatised by e.g. converting a hydroxy group to an alkoxy group, or, by converting one R¹ group to another (e.g. cleaving a the methyl group of —N(CH₃)— to produce —N(H)—). Ester groups may also be hydrolysed to the corresponding carboxylic acid.

The skilled person will appreciate that, to obtain downstream products, the above-mentioned steps may be performed in any order. Further, the reaction steps may be performed separately or conjunctively/sequentially with other reaction steps (for example those described herein) in order to obtain compounds described herein or further derivatives thereof, for example when different functional groups have been modified by different reactions.

The above-mentioned embodiments of the invention are also referred to herein as the “process of the invention”.

Compounds of formula II may be prepared by reduction of a compound of formula III,

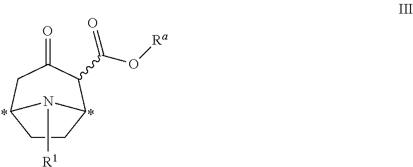

wherein the squiggly line, R^{a} and R¹ are hereinbefore defined, in the presence of an appropriate reducing agent. Most preferably, the reduction reaction is performed on a compound of formula III in which R^{a} represents methyl. The reduction may result in the formation of a single diastereoisomer (or in the two possible cis diastereoisomers), for example, the carbon atom to which the carbonyl group is attached may be reduced in a chemoselective manner, e.g. by catalytic hydrogenation (for example hydrogenation in the presence of H₂ (e.g. a hydrogen atmosphere or nascent hydrogen, e.g. ammonium formate) and a precious metal catalyst (e.g. PtO₂ or Pd/C), in the presence of an appropriate solvent), which may deliver the hydrogen atom from a certain face thereby favoring the cis diastereoisomers of formula IIIA or formula IIIB,

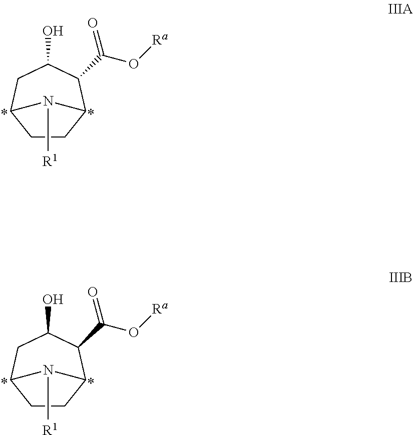

However, in a particular advantageous aspect of the present invention, the reduction need not be chemoselective, and may preferably produce a mixture of cis (syn) and trans (anti) diastereoisomers of formula II. Hence, the reduction may preferably be performed in the presence of a ‘conventional’ reducing agent (i.e. not in the presence of hydrogenation reaction conditions), such as borane (or a complex thereof), LiAlH₄ (or the like) or, preferably, a borohydride, e.g. sodium cyanoborohydride or, preferably, sodium borohydride or lithium borohydride. Any other suitable reducing agents may be employed. However, the skilled person will appreciate that the reducing agent may still need to be chemoselective, in order to prevent reduction of other functional groups in the compound of formula III (e.g. any carboxylic acid or ester group that may be present). For example, if R^{a} is other than hydrogen, then the reducing agent is preferably NaBH₄, which reagent will reduce the carbonyl group to the hydroxy group, but may not reduce the carboxylic acid ester group (or at least will only do so to a minor degree). If the compound of formula III is one in which R^{a} is hydrogen (which may be formed by hydrolysis of the corresponding ester in situ), then NaBH₄ or a stronger reducing agent such as LiBH₄ (or even LiAlH₄) may be employed, as the skilled person will appreciate that the carboxylic acid group is harder to reduce.

Such reductions may be performed in the presence of a suitable solvent, such as an alcoholic solvent (e.g. ethanol or, preferably, methanol). The reduction may be performed at low temperature, for example at below 0° C. (e.g. at below −10° C., such as at about −30° C.). The number of equivalents of reducing agent employed will be known by the person skilled in the art (the reducing agent should supply at least the number of hydrogen atoms required for complete reduction to take place, i.e. where NaBH₄ is employed, at least a quarter of an equivalent is required; however, in practice, at least half or, preferably, at least one equivalent of NaBH₄ is preferred, e.g. at least 1.1 (e.g. at least 1.2), for example about 1.25 equivalents). Preferably, the reducing agent is added in small portions over a period of time, dependent on the scale of the reaction (for example when about 180 g of a tartaric salt of the compound of formula III is to be reduced, then the reducing agent is added in several (e.g. five) portions, which are spaced apart (e.g. by about 1.5 hours)). The primary reason for the slow addition of the reducing agent is to keep the temperature of the reaction mixture low. Preferably, during the addition of the reducing agent, the reaction temperature is retained at about the temperature to which it is initially cooled (e.g. preferably at about −30° C.). The temperature range is preferably kept to within ±10° C. (e.g. at ±5° C.) of the starting temperature. After the reduction reaction is substantially complete, the reaction may be quenched appropriately (e.g. with a source of H⁺ ions, e.g. conc. HCl, which may be pre-cooled). The skilled person will appreciate that a chemoselective reducing agent may need to be employed in order to prevent reduction of other functional groups in the compound of formula III (e.g. any carboxylic acid or ester group that may be present).

Compounds of formula III in which R^{a} is hydrogen may be prepared by hydrolysis of a corresponding ester, i.e. from a corresponding compound of formula III in which R^{a} is other than hydrogen.

Particularly preferred compounds of formula III that may be reduced to compounds of formula II include those in which R^{a} represents C₁₋₃ (e.g. C₁₋₂) alkyl groups such as methyl or, preferably, ethyl.

In the process of the invention, a salt of the compound of formula III (for example a tartaric acid salt, which is a single enantiomer obtained by resolution) may be employed, which may first be neutralised under standard conditions, for example in the presence of a suitable base, for instance a weak base such as an alkali metal based base (e.g. CH₃ONa, K₂CO₃, K₃PO₄, t-BuONa, t-BuOK or, preferably, Na₂CO₃) in an appropriate solvent, such as water, or a stronger base, for example in the presence of aqueous sodium hydroxide solution, which may be between 10 and 50% w/w, e.g. between 15 and 30% w/w, e.g. 20% w/w). As discussed hereinbefore, the compound of formula III need not be of an ee near 100% (but may be of a lower ee).

It should be appreciated that the purified compound of formula I so formed by the process of the invention may also contain materials other than those specified above.

This product may be further purified using any suitable separation/purification technique or combination of techniques including further crystallisation, distillation, phase separation, adsorption, e.g. using molecular sieves and/or activated carbon, and scrubbing.

Compounds described herein, and derivatives thereof (e.g. protected derivatives), may be commercially available, are known in the literature or may be obtained by conventional synthetic procedures, in accordance with known techniques, from readily available starting materials using appropriate reagents and reaction conditions.

Substituents on compounds described herein, or any relevant intermediate compounds to such compounds (or salts, solvates or derivatives thereof), may be modified one or more times, before, after or during the processes described above by way of methods that are well known to those skilled in the art. Examples of such methods include substitutions, reductions, oxidations, alkylations, acylations, hydrolyses, esterifications, etherifications, halogenations, nitrations, diazotizations or combinations of such methods.

It is stated herein that functional groups may be protected. It will also be appreciated by those skilled in the art that, in the processes described above, other functional groups of intermediate compounds may be, or may need to be, protected by protecting groups.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective

Groups in Organic Synthesis”, 3^{rd} edition, T. W. Greene & P. G. M. Wutz, Wiley-Interscience (1999).

In a further aspect of the invention, there is provided a process for the preparation of a pharmaceutical formulation of a compound of formula I or a product downstream of a compound of formula I (e.g. a compound of formula IA, IB, IC, ID or derivatives thereof, e.g. esters or hydroxy derivatives, such as alkoxy groups), or a salt of any of these compounds, which process comprises a process as described herein for the preparation of the relevant compound, and bringing into association the compound so formed, with (a) pharmaceutically-acceptable excipient(s), adjuvant(s), diluent(s) and/or carrier(s).

The processes described herein may be operated as a batch process or operated as a continuous process and may be conducted on any scale.

The process of the invention may also have the advantage that the compound of formula I is produced in higher yield, in higher purity, in higher selectivity (e.g. higher enantioselectivity), in less time (for example due to an increased rate of reaction compared to the prior art), with better efficiency, in a more convenient (i.e. easy to handle) form, from more convenient (i.e. easy to handle) precursors, at a lower cost and/or with less usage and/or wastage of materials (including reagents and solvents) compared to the procedures disclosed in the prior art.

In particular, the process of the invention has the advantage that, starting from either enantiomer of the intermediate compound of formula III (i.e. the carbalkoxytropinone) the corresponding single enantiomer of the compound of formula I may be produced. Advantageously, the formation of the single enantiomer of the compound of formula I need not be dependent upon the configuration at the carbon atom bearing the —OH or —C(O)OR^{a} group in the compound of formula II (produced by reduction of the carbonyl group of the compound of formula III), i.e. the precursor compound of formula II may exist as diastereoisomers without effecting the process of the invention. Put another way, the process of the invention may have the advantage that single or even two (e.g. the cis) diastereoisomers of the compound of formula II need not be employed (but the reaction may be performed on the cis and trans diastereoisomers), i.e. certain diastereoisomers of the compound of formula II (or an ester thereof) are not necessary as a precursor in order to provide a single enantiomer of a compound of formula I (after an elimination reaction) in reasonable yields (e.g. without leaving a substantial amount of a diastereoisomer of a compound of formula II unreacted).

Furthermore, the process of the invention has the advantage that either enantiomer of the compound of formula I may be produced, starting from the relevant enantiomer of any precursor (i.e. compound of formula II or III). This is in stark contrast to any prior art processes, which for example may only obtain racemic compounds of formula I (if starting from racemic starting materials) or a certain enantiomer of the compound of formula I (for example, only the R-enantiomer may be obtained, if using a ‘chiral pool’ method starting from e.g. cocaine).

In general, the processes described herein, may also have the advantage that the compounds of formula I may be produced in a manner that utilises fewer reagents and/or solvents, and/or requires fewer reaction steps (e.g. distinct/separate reaction steps) compared to processes disclosed in the prior art.

The following examples are merely illustrative examples of the processes of the invention described herein.

All equipment, reagents and solvents used were standard laboratory equipment, e.g. glassware, heating apparatus and HPLC apparatus.

### EXAMPLES

### Example 1

Synthesis of R-anhydroecqonine acid HBr salt

To a solution of sodium carbonate (116.3 g, 1.1 mol) in water (650 g) was added R-2-carboxyethyltropinone tartaric acid salt (180.6 g, 0.50 mol, 98%-ee) portion wise. The solution was extracted with toluene (268 g). The cut (i.e. separated) aqueous phase was pH adjusted with sodium hydroxide (150 g, 20% w/w solution) and then again extracted with toluene (171 g). The toluene phases were pooled and filtered through celite (7 g). The solvent was stripped off under vacuum (50° C. on the jacket). To the oily residue was added methanol (592 g) and the mixture was cooled to −30° C. Sodium borohydride (23.6 g, 0.625 mol) was added in 5 equally sized portions with 1.5 hour between each portion. The temperature was kept at −30±5° C. until complete reaction (<5% of starting material remained). The reaction mixture was quenched with a pre-cooled mixture of concentrated hydrochloric acid (42 g) and water (42 g) at such a rate that the temperature was kept below 20° C. After quench the pH was adjusted to pH<6 with a small portion of concentrated hydrochloric acid and stirred for 10 min. The pH was adjusted to 7 with sodium hydroxide (20% w/w solution) and the methanol was stripped off under vacuum. The pH was adjusted to 10.8 with sodium hydroxide (105 g, 20% w/w solution) and the mixture was extracted with ethyl acetate (160 g). The cut aqueous phase was pH adjusted to pH 11 and again extracted with ethyl acetate (160 g). The ethyl acetate phases were pooled and the solvent was stripped off under vacuum. To the oily residue was added hydrobromic acid (48%, 253 g, 1.50 mol) and the mixture brought to reflux. After 1 hour at reflux ethanol was distilled off until less then 1% of the intermediate product remained. The mixture was cooled and propionic acid (371 g, 5.0 mol) was charged. The vessel was closed and the mixture heated to 165±5° C. for 8 hours when less then 5% of the intermediate product remained. The mixture was cooled to 100° C. and water and propionic acid was distilled off. When a total volume of 300mL had been distilled off the reaction mixture was cooled to 22° C. To the resulting slurry was dosed a mixture of ethyl acetate (640 g) and ethanol (110 g) during 30 min. The slurry was then cooled to 5° C. and stirred for 30 min. where after the crystals were filtered off and washed with ethyl acetate (132 g). The crystals were dried over night affording 69.5 g, 56% yield, of R-anhydroecgonine HBr salt. HPLC purity >99.9%.

Synthesis of R-anhydroecqonine ethyl ester

To R-anhydroecgonine HBr salt (69.0 g, 0.28 mol) was charged ethanol (252 g) and sulfuric acid (30.0 g, 0.31 mol). The mixture was brought to reflux and kept there for 2 hrs (86% conversion of the starting material). The vessel was set up for distillation and 260 mL solvent was distilled off. Ethanol (252 g) was again added and 250 mL solvent was distilled off. The mixture was cooled to 20° C. and there after quenched to a solution of sodium carbonate (64 g, 0.60 mol) and water (587 g) while keeping the temperature <25° C. The mixture was concentrated under vacuum (jacket temperature set to 50° C.). When 130 g solvent had been stripped off toluene (140 g) was added together with Nuchar charcoal (2.7 g). The mixture was vigorously agitated for 30 min at 20° C. and then filtered through celite (2.7 g). The layers were cut and the aqueous layer was pH adjusted with sodium hydroxide (20% w/w solution) to pH 10 and again extracted with toluene (140 g). The toluene phases were pooled and concentrated under vacuum (jacket set to 50° C.) until no more toluene distilled off, affording 50.9 g R-anhydroecgonine ethyl ester in 92% yield (corrected for assay 98%). HPLC purity >99.9%, enantiomeric excess >99.8%.

### Example 2

The S-anhydroecgonine ethyl ester was prepared in accordance with the procedures described in Example 1, providing similar yields and enantioselectivity.

### Example 3

Compounds of the invention (e.g. those prepared by the processes described herein, such as compounds of formulae I, IA, IB and/or IC) may be formulated into a pharmaceutically acceptable formulation using standard procedures.

For example, there is provided a process for preparing a pharmaceutical formulation comprising compounds of the invention (e.g. those prepared by the processes described herein, such as compounds of formulae I, IA, IB and/or IC), or a salt thereof, which process is characterised in that it includes as a process step a process as hereinbefore defined. The skilled person will know what such pharmaceutical formulations will comprise/consist of (e.g. a mixture of active ingredient and pharmaceutically acceptable excipient, adjuvant, diluent and/or carrier).

There is further provided a process for the preparation of a pharmaceutical formulation comprising compounds of the invention (e.g. those prepared by the processes described herein, such as compounds of formulae I, IA, IB and/or IC), which process comprises bringing into association the active compound, or a pharmaceutically acceptable salt thereof (which may be formed by a process as hereinbefore described), with (a) pharmaceutically acceptable excipient(s), adjuvant(s), diluent(s) and/or carrier(s).

1. A process for the preparation of a single enantiomer of anhydroecgonine (of formula I): 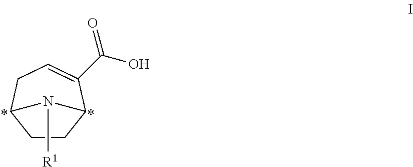
or a salt thereof, wherein:
R¹ represents hydrogen or optionally substituted C₁₋₁₂ alkyl;
the asterisks each denote a chiral center that has a certain configuration, by elimination (and, if required, hydrolysis) of a compound of formula II, 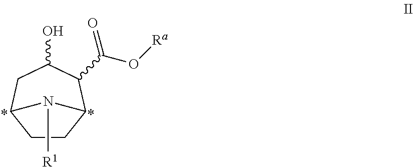
wherein:
the asterisks each denote a chiral center that has a certain configuration;
the squiggly lines (attached to the OH group and to the C(O)OR^{a} group) each denote a bond that is attached to a chiral center that can be of R- or S-configuration;
R^{a} represents: hydrogen; optionally substituted aryl or heteroaryl; or, preferably, optionally substituted C₁₋₁₂ alkyl; and
R¹ is as defined above,
in the presence of a strong acid and a carboxylic acid.

2. A process as claimed in claim 1, which proceeds via an intermediate of formula IIA, 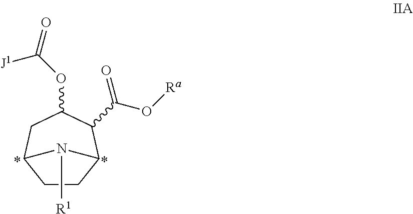
in which J¹ represents the counterpart to the carboxylic acid functional group, and R^{a} and R¹ are as defined in claim 1.

3. A process as claimed in claim 1, wherein the carboxylic acid is a compound of formula IIB,
J¹-COOH IIB
in which J¹ represents hydrogen or C₁₋₁₂ alkyl, which is optionally substituted by one or more substituents selected from carboxy, halo and phenyl (optionally substituted by one or more halo atoms).

4. A process as claimed in claim 3, wherein J¹ represents unsubstituted C₁₋₃ alkyl.

5. A process as claimed in claim 4, wherein the J¹ represents ethyl (and hence the carboxylic acid is propionic acid).

6. A process as claimed in claim 1, wherein the strong acid is a hydrogen halide.

7. A process as claimed in claim 6, wherein the acid is HBr.

8. A process as claimed in claim 1, wherein R^{a} represents C₁₋₃ alkyl.

9. A process as claimed in claim 1, wherein R¹ represents hydrogen or C₁₋₃ alkyl.

10. A crystalline compound formed by an association between:
(i) a compound of formula I as defined in claim 1; and
(ii) an acid
wherein moiety (i) and (ii) are in a ratio of about 1:1; and/or

11. A compound formed by an association between:
(i) a compound of formula I as defined in claim 1; and
(ii) HI or HBr.

12. A crystalline compound as claimed in claim 10, wherein moiety (ii) is a hydrogen halide.

13. A crystalline compound as claimed in claim 12, wherein the hydrogen halide is HI or HBr.

14. A compound as claimed in claim 11, wherein moiety (i) and (ii) are in a ratio of about 1:1.

15. A compound as claimed in claim 11, wherein the compound is in solid form.

16. A compound as claimed in claim 15, wherein the compound is in crystalline form.

17. A crystalline compound as claimed in claim 10, which is a salt.

18. A process for the preparation of a compound as defined in claim 10, which comprises:
(i) bringing into association or contacting a compound of formula I as defined in claim 1 and the acid, in a solvent; and
(ii) crystallization or precipitation of the compound so formed, in the solvent.

19. A process as claimed in claim 18, wherein the compound to be formed is an association between the compound of formula I and HI or HBr, and the process comprises:
(i) a process for the preparation of the HI or HBr salt of a compound of formula I as claimed in claim 7, in the presence of a carboxylic acid;
(ii) crystallization or precipitation of the HBr salt so formed, in carboxylic acid.

20. (canceled)

21. A process as claimed in claim 18, further comprising at least one step of recrystallization.

22. (−)-Anhydroecgonine, which has an ee of greater than 95%.

23. A single enantiomer of anhydroecgonine, characterized in that the HPLC purity is greater than 99%.

24. A process comprising preparation of a compound of formula II, by reduction of a compound of formula III, 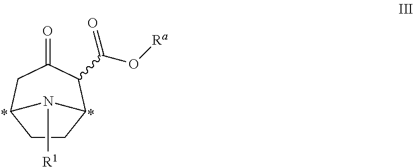
wherein R^{a}, R¹ and the squiggly line are as defined in claim 1, followed by a process as claimed in any one of claim 1 to 9, 20 or 21 (as dependent on claim 20).

25. A process wherein the process of claim 1 is followed by:
(A) conversion to a compound of formula IA, 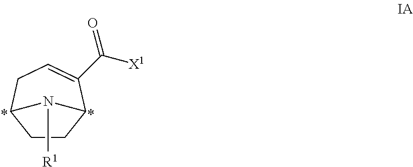
in which Xⁱ represents:
(i) —OR^{b1};
(ii) —N(R^{b2})R^{b3},
R_{b1} represents optionally substituted aryl or heteroaryl;
R^{b2} and R^{b3} independently represent hydrogen; optionally substituted C₁₋₁₂ alkyl; or optionally substituted aryl or heteroaryl;
R¹ is as defined in claim 1; or
(B) conversion to a compound of formula IB, 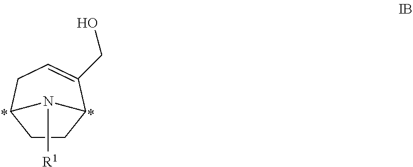
by an appropriate reduction (of a compound of formula I or an ester of formula IA (in which X¹ represents —OR^{b1})).

26. A process for the preparation of a compound of formula IC, 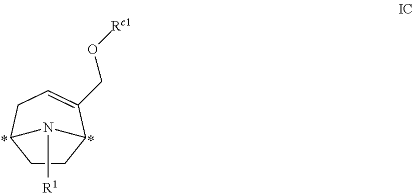
wherein R^{c1} represents optionally substituted C₁₋₁₂ alkyl, and R¹ is as defined in claim 1, which comprises a process for the preparation of a compound of formula IB as claimed in claim 25, followed by alkylation in the presence of a compound of formula ICA,
L¹-R^{c1} ICA
wherein L¹ represents a suitable leaving group.

27. A process for the preparation of a pharmaceutical formulation comprising a compound of formula I, or a salt thereof, which process comprises:
(i) bringing into association a compound of claim 10, with (a) pharmaceutically-acceptable excipient(s), adjuvant(s), diluent(s) or carrier(s);

28. (canceled)

29. A compound as claimed in claim 11, which is a salt.

30. A process for the preparation of a pharmaceutical formulation comprising a compound, or, a salt thereof, of formula I comprising preparing a compound of formula I according to the process of claim 1 followed by bringing into association the compound, or a salt thereof, with (a) pharmaceutically-acceptable excipient(s), adjuvant(s), diluent(s) or carrier(s).
